(19) United States
(12) Patent Application Publication
(10) Pub. No.: US 2021/0169840 A1
(43) Pub. Date: Jun. 10, 2021

(54) COMPOSITION AND METHOD OF AN IRON SUPPLEMENT

(71) Applicant: Laurence Stanley Kubiak, Wellington (NZ)

(72) Inventor: Laurence Stanley Kubiak, Wellington (NZ)

(21) Appl. No.: 17/116,086

(22) Filed: Dec. 9, 2020

Related U.S. Application Data
(60) Provisional application No. 62/945,427, filed on Dec. 9, 2019.

(51) Int. Cl. A61K 31/295; A61K 36/906; A61K 31/375; A61K 9/20; A61K 9/48; A61K 9/68; A61K 31/191; A61K 31/194; A61K 31/095; A23L 33/00; A23L 33/105; A23L 33/15

(52) U.S. Cl. CPC A61K 31/295; A61K 36/906; A61K 31/375; A61K 9/20; A61K 9/48; A23L 33/15; A61K 31/191; A61K 31/194; A61K 31/095; A23L 33/30; A23L 33/105; A61K 9/0058

(57) ABSTRACT

A nutritional supplement composition, is disclosed. The nutritional supplement composition includes one or more vitamins and mixtures. The one or more vitamins and mixtures are selected from the group consisting of an iron supplement comprising a combination of active ingredients. The active ingredients include carbonyl iron. The active ingredients include vitamin C. The active ingredients include ginger.

### FIELD OF THE INVENTION

The present disclosure generally relates to iron supplements, vitamins, and other iron-based supplements for healthy consumption. More specifically, the present disclosure provides for an improved iron absorption and a method thereof that eliminates the side effects normally associated with iron supplements.

### BACKGROUND

The human body requires a multitude of different vitamins and nutrients in order to ensure proper functionality, continual growth, and a strong immune system. Humans often have to supplement their daily intake of food, water, and other substances with additional vitamins and supplements in order to ensure that their bodies continue to function in good working order. One of the many vitamins that are of essential importance is iron.

Iron is an essential mineral that the body needs for growth and development. Your body uses iron to make hemoglobin, a protein in red blood cells that carries oxygen from the lungs to all parts of the body, and myoglobin, a protein that provides oxygen to muscles. Your body also needs iron to make some hormones. Essentially, iron helps transport oxygen through the body in order to ensure that your muscles and vital organs have enough oxygen in order to properly function effectively.

A shortage of iron in the blood can lead to a range of serious health problems, including iron deficiency anemia. Around 10 million people in the United States have low iron levels, and roughly 5 million of these have been diagnosed with iron deficiency anemia. Without enough iron, your body can't produce enough of a substance in red blood cells that enables them to carry hemoglobin or oxygen. As a result, iron deficiency anemia may leave you tired and short of breath. Iron deficiencies can often be corrected by taking iron supplements.

Iron supplements are necessary for some users. However, iron supplements often are not easily absorbed into the body. This problem is exacerbated by the use of iron salts (e.g., ferrous sulfate, ferrous fumarate, ferrous gluconate and ferrous succinate) in the iron supplements. The issue with the use of iron salts in iron supplements is they do not absorb well compared to other iron compositions. This makes manufacturers who use iron salt in their supplements increase the dose to ensure users are receiving enough iron. Manufacturers typically increase the dose in multiples of 3-4 the elemental iron content, and it is common that the doses are increased by even higher multiples. These higher doses of the iron salts accentuate the many side effects of the iron supplements.

Additionally, iron supplements are known to usually cause numerous side effects. Iron supplement side effects may include nausea, digestive problems (both constipation and diarrhea, depending on the user), and epigastric problems (e.g., heartburn, indigestion, upset or sore stomach). These side effects are worsened by the increased dose of the iron salts in the typical iron supplement. Iron is also known to have low absorption rates due to the user's diet because some foods interfere with the rate of absorption. Coffee, tea, and dairy products are all known to affect the absorption of iron, but they are popular dietary items that most people will not voluntarily avoid. Typically, iron supplements are taken for a variety of reasons which may include anemia, to reduce fatigue, to improve muscle strength, to boost immunity, and to improve concentration. However, as mentioned herein iron is not easily absorbed into the body, which makes treating these issues more difficult.

It is therefore the intent of the present invention to solve the aforementioned problems, and issues, by providing a solution-based iron supplement. The iron supplement described herein, provides the advantage of having better absorption capability and reduces the multiple side effects iron supplements usually cause.

The aspects or the problems and the associated solutions presented in this section are not approaches that have been previously conceived or pursued. Therefore, unless otherwise indicated, it should not be assumed that any of the approaches presented in this section qualify as prior art merely by virtue of their presence in this section of the application.

### SUMMARY OF THE INVENTION

The present disclosure provides for an improved iron absorption and a method thereof. The iron absorption can be implemented via a supplement that is ingested within the body. Accordingly, iron supplement containing a combination of active ingredients such as carbonyl iron, vitamin C, and ginger with an inactive flow agent ingredient such as stearate is provided.

In an aspect, an iron supplement is provided having carbonyl iron as the source of iron. Carbonyl iron is absorbed by body at a much higher rate (typically a 70% absorption for pure carbonyl iron) compared to other iron sources. This means an iron supplement dose using carbonyl iron may be much lower compared to the dose when using an iron salt. Carbonyl iron is known to be better absorbed by the body because it is microparticles of elemental iron. In some embodiments, elemental irons can incorporate elements other than iron salt. Thus, an advantage of using carbonyl iron in the iron supplement is a reduction in side effects due to the lower dose necessary for a daily value of iron.

In another aspect, an iron supplement is provided having ginger as an active ingredient. The carbonyl iron reduces side effects compared to the iron salts, but iron is still an irritant to the human digestive tract. The ginger extract within the supplement further reduces side effects, such as nausea and vomiting. Ginger also aids digestion and is effective against epigastric problems. It is known that stomach mucosa cushions the delicate stomach lining from contents of the stomach and typically plays a role in the absorption of some nutrients. This is done by holding the stomach contents in suspension until they can be fully absorbed by the body. Ginger extract increases the production of gastric acid and stimulates the production of stomach mucosa. The increased production of gastric acid is necessary for efficient digestion of food, but more importantly helps with the absorption of iron in the mucous membranes of the duodenum. The carbonyl iron becomes more absorbable through its reaction with the additional gastric acid produced by the ginger extract. Thus, an advantage is higher iron absorption rates due to the additional gastric acid in the stomach and small intestine.

In another aspect, an iron supplement is provided having vitamin C as an active ingredient. Vitamin C, also referred to as ascorbic acid, is a water-soluble vitamin, is a reducing agent, and reacts with the carbonyl iron. The vitamin C in the iron supplement combines with the carbonyl iron to increase the solubility of iron in the body, resulting in increased absorption in the mucous membranes of the duodenum, which is the first section of the small intestine immediately after the stomach. The vitamin C combines with the iron to form a chelate. A chelate is a compound which transforms an inorganic form, a mineral or an element, into an organic one, which can then be transported across the intestinal wall. This process is how all inorganic minerals and elements are absorbed by the body. The chelate is a substance at an acidic pH level that remains soluble at the alkaline pH level of the duodenum. Vitamin C also enables the mobilization of iron from storage within the body. The amount of iron absorbed is directly proportionate to the amount of vitamin C consumed simultaneously.

Additionally, vitamin C reduces ferric iron to ferrous iron, which are different states of oxidation. This is also beneficial because ferric iron cannot be absorbed by the body, while ferrous iron can be absorbed through the stomach and intestinal mucosa. Vitamin C also has been shown to increase the formation of serum iron (iron in the blood), ferritin (the protein that controls the release of iron into the blood), and the concentration of hemoglobin (the protein which transports oxygen through the blood). Vitamin C may also counteract the negative compounds that other dietary items create that inhibit iron absorption. Meaning the vitamin C prevents the formation of insoluble and therefore unabsorbable iron compounds within the body. Thus, an advantage is better iron absorption into the body due to the combination of carbonyl iron and vitamin C.

In another aspect, an iron supplement is provided having an administration protocol, wherein the iron supplement is asked to be consumed immediately before going to sleep for the night. The administration protocol allows for minimal interference from dietary choices with the iron absorption. The substances that are most likely to interfere with iron absorption (i.e., coffee, tea, dairy products) are most commonly consumed well before bedtime. The administration protocol allows for most of the absorption to take place overnight while the user sleeps. Since the user is sleeping while the absorption is taking place, the user would not feel any remaining side effects of the iron absorption. Thus, an advantage is the user will face less competition between iron absorption and other foods being digested. Another advantage is that the user may sleep through additional side effects of consuming the iron supplement.

The administration protocol further allows for the iron to be more fully absorbed by morning and will be bioavailable in the user's blood stream first thing in the morning. This allows the user to avoid the fatigue and low energy, which commonly occurs when iron levels are low. These effects also occur because there is a gap between taking iron supplements and when the iron becomes bioavailable. Thus, another advantage of the administration protocol is the bioavailability of the iron when the user wakes up.

In another aspect, an iron supplement is provided having an effective minimum dose. The composition of the active ingredients of the iron supplement may be kept near the effective minimum dose for each ingredient to limit the user from having too much of any given substance in their system. Using the effective minimum doses for the iron supplement allows the user to also consume other supplements, which is typically done, without having too much of the active ingredients in their system. Thus, an advantage of providing effective minimum doses in the iron supplement is that the user may not experience issues if they are also consuming other supplements with the active ingredients of the iron supplement.

A nutritional supplement composition, is disclosed. The nutritional supplement composition includes one or more vitamins and mixtures. The one or more vitamins and mixtures are selected from the group consisting of an iron supplement comprising a combination of active ingredients. The active ingredients include carbonyl iron. The active ingredients include vitamin C. The active ingredients include ginger.

In one embodiment, the inactive flow agent ingredient is stearate.

In one embodiment, the supplement includes 8 mg of carbonyl iron, 70 mg of vitamin C, and 250 mg of ginger.

In one embodiment, the supplement includes an amount of carbonyl iron is selected from an amount between 2 milligrams (mg) and 45 mg.

In one embodiment, the vitamin C is combined with an elemental iron.

In one embodiment, the elemental iron is 200 milligrams (mg).

In one embodiment, the ratio of vitamin C to elemental iron is selected from a range between 7:1 and 9:1.

In one embodiment, the elemental iron is selected from one or more of ferrous sulfate, ferrous fumarate, ferrous glucomate and ferrous succinate.

In one embodiment, the amount of ginger in the iron supplement is selected from a range from 250 mg to 500 mg.

In one embodiment, the iron supplement may be consumed orally as a capsule containing the combination of carbonyl iron, vitamin C, and ginger.

In one embodiment, the iron supplement is one or more of a tablet, a capsule, a suspension, a gum, or an effervescent tablet.

In one embodiment, the ginger is a ginger extract.

In one embodiment, the ginger extract is combined with the carbonyl iron comprising of a ratio of 10:1.

In one embodiment, the ginger extract is configured as a ginger component within the iron supplement to produce additional gastric acid.

In one embodiment, the vitamin C is water-soluble and a reducing agent.

In one embodiment, the vitamin C is an amount between 30 milligrams (mg), and 70 mg.

The above aspects or examples and advantages, as well as other aspects or examples and advantages, will become apparent from the ensuing description. This summary is provided merely for purposes of summarizing some example embodiments, so as to provide a basic understanding of some aspects of the subject matter described herein.

Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### DETAILED DESCRIPTION

Some detailed example embodiments are disclosed herein. However, specific structural and functional details disclosed herein are merely representative for purposes of describing example embodiments. Example embodiments may, however, be embodied in many alternate forms and should not be construed as limited to only the embodiments set forth herein.

The invention may be embodied in other specific forms without departing from the spirit of essential characteristics thereof. The present embodiments therefore to be considered in all respects as illustrative and not restrictive, the scope of the invention being indicated by the appended claims rather than by the foregoing description, and all changes which come within the meaning and range of equivalency of the claims are therefore intended to be embraced therein.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The following description is not to be taken in a limiting sense, but is made merely for the purpose of describing the general principles of exemplary embodiments. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

An iron supplement containing a combination of active ingredients such as carbonyl iron, vitamin C, and ginger with an inactive flow agent ingredient such as stearate is provided.

As it will be described in more detail hereinafter, the combination of these three active ingredients may work together to increase iron absorption in the human body and decrease possible side effects of the consumed iron. For example, the iron supplement may contain approximately 8 mg of carbonyl iron, 70 mg of vitamin C, and 250 mg of ginger.

Typically, when using carbonyl iron one capsule contains approximately 8 mg, which may supply the normal supplemental dose required by a man or a post-menopause woman, while two capsules containing a total of 16 mg of carbonyl iron may supply the normal supplemental dose required by a woman who is between puberty and menopause. However, the recommended daily average dose for carbonyl iron usually is 8 mg for men and 18 mg for women. For example, a woman who may be suffering from low energy or a troublesome period may take another capsule (8 mg), bringing the total estimated supplemental dose to 24 mg. This is still well within the tolerable upper intake level, which is 45 mg a day for men and women. However, it is known that if a woman is regularly taking more than 24 mg of elemental iron and is not pregnant, she should consult her doctor. Having approximately 8 mg of the carbonyl iron in the iron supplement may allow the user to receive the effective minimum dose, but this also allows the user to take additional supplements if necessary.

Normally, when using vitamin C with iron salts 200 mg may be needed per 30 mg of elemental iron to enhance the absorption, which is a ratio of almost 7:1. In many instances, iron salts can also be other elemental irons such as carbonyl iron. The ratio of vitamin C and carbonyl iron may be increased, but effectiveness has been shown to decrease when the ratio nears 9:1. The amount of vitamin C in the iron supplement may be larger than the 70 mg. However, it may be considered to stay in the range of the minimum effective dose because users often take multiple supplements simultaneously, meaning the user may be consuming higher quantities of these active ingredients.

Usually when using ginger, 250 mg is the minimum effective dose for menstrual cramps and morning sickness, while a maximum dose is approximately 500 mg. In an example, the amount of ginger in the iron supplement may range from approximately 250 mg to 500 mg because of the many benefits ginger has. However, as mentioned the 250 mg may be used because it is the effective minimum dose especially when considering a user may be getting more ginger elsewhere from their diet. The combination of carbonyl iron, vitamin C, and ginger in the iron supplement allows for a decrease in the many side effects typical iron supplements cause. The iron supplement may be consumed orally as a capsule containing the combination of carbonyl iron, vitamin C, and ginger.

In an example, the iron supplement may be manufactured in any of the standard formats (e.g., tablet, capsule, suspension, gum, effervescent tablet) for the user's consumption. Inert flow agents such as stearate may also be included in the manufacturing process to ensure the doses are accurate. The flow agent prevents the active ingredients from clumping together and becoming suck in the manufacturing equipment, which may inhibit the dose from being accurate during manufacturing. The flow agent affects neither an administration protocol nor the active formulation because it is an added inactive ingredient.

Iron supplements are known to usually cause numerous side effects, which may include nausea, digestive problems (both constipation and diarrhea, depending on the subject), and epigastric problems (e.g., heartburn, indigestion, upset or sore stomach). These side effects are worsened by the increased dose of the iron salts in a typical iron supplement. Iron is also known to have low absorption rates due to the user's diet, which some interfere with the rate of absorption. Coffee, tea, and dairy products are all known to interfere with the absorption of iron, but they are popular dietary items that most people will not voluntarily avoid. The use of the carbonyl iron may decrease the amount of side effects because it may be used in a lower dose, while the combination of ginger, vitamin C, and an administration protocol may decrease the remaining side effects that may occur.

The carbonyl iron may be better absorbed by the human body compared to iron salts because carbonyl iron are microparticles of elemental iron, while iron salts are iron sulfates which is not pure iron. Carbonyl iron may be absorbed by body at a much higher rate (typically a 70% absorption for pure carbonyl iron), which means dose can be much lower compared to the doses when using an iron salt. This means there may be reduced side effects due to the lower dose necessary to reach a daily value of iron. Carbonyl iron is typically not used in iron supplements because iron salts are water soluble. However, even though iron salts may be water soluble they are not absorbed well in the human body. Thus, for iron salts to work as an iron supplement the dose needs to be higher than what is typically needed because of the low absorption rate.

As mentioned, iron salts are used instead of carbonyl iron, but the side effects of iron consumption are exacerbated by the use of iron salts (e.g., ferrous sulfate, ferrous fumarate, ferrous glucomate and ferrous succinate) in supplements. The issue with the use of iron salts in iron supplements is they do not absorb well compared to other iron compositions. This makes manufactures who use iron salt in their supplements increase the dose to ensure users are receiving enough iron. Manufacturers typically increase the dose in multiples of 3-4 the elemental iron content, and it is common that the doses are increased by even higher multiples. These higher doses of the iron salts accentuate the many side effects of the iron supplements. Carbonyl iron may be used instead of irons salts to allow for a lower dose, which may also lower the amount of side effects the user would experience. There may be a lower dose of carbonyl iron compared to the usual dose for iron salts, but the iron supplement using carbonyl iron may still provide the daily value of iron to the user.

Vitamin C may be used in the iron supplement because it increases the iron absorption rate and counteracts negative effects other dietary items might have on the iron absorption rate. The vitamin C used in the iron supplement may increase absorption because vitamin C is a water-soluble vitamin and it is a reducing agent. The vitamin C coupled with the carbonyl iron may increase the solubility of iron in the small intestine, resulting in increased absorption in the mucous membranes of the duodenum, which is the first section of the small intestine immediately after the stomach where the iron is absorbed. As mentioned, vitamin C also counteracts the negative effects of other dietary items consumed that inhibit iron absorption. The iron absorption in the body is improved due to the combination of carbonyl iron and vitamin C. To receive the benefits described herein, the vitamin C should be consumed at the same time as the carbonyl iron, which is why they are coupled together within the iron supplement. The dose of vitamin C is also larger compared to the dose of carbonyl iron within the supplement because of their proportional relationship. This means the rate of iron absorption may be directly related to amount of vitamin C compared to carbonyl iron present in the iron supplement. However, it is known that this increased absorption rate does not increase further once the ratio of vitamin C to carbonyl reaches 9:1.

The ginger, or ginger extract, may be used in the iron supplement to decrease potential side effects of the carbonyl iron. As described herein, the carbonyl iron reduces side effects compared to the iron salts, but iron is still an irritant to the human digestive tract. The ginger extract within the supplement reduces side effects, such as nausea and vomiting. Ginger also aids digestion and is effective against epigastric problems. The stomach mucosa cushions the delicate stomach lining from contents of the stomach and typically plays a role in the absorption of some nutrients by holding them in suspension until they can be absorbed by the body. Iron is absorbed in the small intestine, while the ginger extract increases the production of gastric acid and stimulates the production of stomach mucosa. The increased production of gastric acid may be necessary for efficient digestion of food, but also may be necessary for reacting to the carbonyl iron to be better absorbed in the small intestine. Having an increased production of gastric acid may be beneficial because low gastric acid levels are linked to impaired digestion, increased risk of infection, and a reduction of the body's ability to absorb nutrition from food. The symptoms of low gastric acid include nausea, digestive problems (both constipation and diarrhea), and epigastric problems (heartburn, indigestion, upset or sore stomach). Carbonyl iron also may become more absorbable through its reaction with additional gastric acid produced by the ginger extract within the iron supplement. For example, 10:1 ginger extract may be used as the ginger component within the iron supplement to produce the additional gastric acid.

The iron supplement would have an administration protocol that may instruct the user to consume the iron supplement immediately prior to going to sleep for the night. The administration protocol allows for the user to absorb the supplement uninterrupted while sleeping. Having the administration protocol allows for minimal interference from dietary choices with the iron absorption. As mentioned herein, the substances that are most likely to interfere with iron absorption (e.g., coffee, tea, and dairy products) are most commonly consumed well before bedtime, which if the administration protocol is followed these substances may not interfere with the iron absorption. The administration protocol allows for most of the iron absorption to take place overnight while the user sleeps. This means the digestive tract of the user may face less competition between iron absorption and other foods being digested. The iron may be fully absorbed by morning and therefore may be bioavailable in the user's blood stream at that time. The bioavailable iron allows the user to avoid fatigue and low energy, which are characteristic both of low iron levels and the typical gap between taking an iron supplement and when the iron becomes bioavailable. The administration protocol also allows the user to sleep through any remaining side effects the ginger does not resolve.

The effects of the ginger extract in the supplement described herein may not happen instantaneously because the ginger requires time to digest and to begin acid production. The administration protocol takes this into consideration and instructs the user to consume the supplement prior to falling asleep for the night. The administration protocol allows to the user to be asleep during the time the side effects might occur. The administration protocol also allows the user's body to have time to react to the ginger extract and begin production of the acids to further reduce the ongoing side effects while sleeping.

Example embodiments having thus been described, it will be obvious that the same may be varied in many ways. Such variations are not to be regarded as a departure from the intended spirit and scope of example embodiments, and all such modifications as would be obvious to one skilled in the art are intended to be included within the scope of the following claims.

Although the present invention has been described in terms of various embodiments, it is not intended that the invention be limited to these embodiments. Modification within the spirit of the invention will be apparent to those skilled in the art.

It is additionally noted and anticipated that although the device is shown in its most simple form, various components and aspects of the device may be differently shaped or modified when forming the invention herein. As such those skilled in the art will appreciate the descriptions and depictions set forth in this disclosure or merely meant to portray examples of preferred modes within the overall scope and intent of the invention and are not to be considered limiting in any manner. While all of the fundamental characteristics and features of the invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the scope of the invention.

What is claimed is:

1. A nutritional supplement composition comprising one or more vitamins and mixtures selected from the group consisting of an iron supplement comprising a combination of active ingredients comprising:
carbonyl iron,
vitamin C, and
ginger.

2. The nutritional supplement of claim 2, wherein the inactive flow agent ingredient is stearate.

3. The nutritional supplement of claim 1, comprising of 8 mg of carbonyl iron, 70 mg of vitamin C, and 250 mg of ginger.

4. The nutritional supplement of claim 1, wherein an amount of carbonyl iron is selected from an amount between 8 milligrams (mg) and 45 mg.

5. The nutritional supplement of claim 1, wherein the vitamin C is combined with elemental iron.

6. The nutritional supplement of claim 5, wherein the elemental iron is selected from a range of 45 milligrams to (mg) 200 mg.

7. The nutritional supplement of claim 5, wherein the ratio of vitamin C to elemental iron is selected from a range between 7:1 and 9:1.

8. The nutritional supplement of claim 5, wherein the elemental iron is selected from one or more of ferrous sulfate, ferrous fumarate, ferrous gluconate and ferrous succinate.

9. The nutritional supplement of claim 1, wherein the amount of ginger in the iron supplement is selected from a range from 250 mg to 500 mg.

10. The nutritional supplement of claim 1, wherein the iron supplement may be consumed orally as a capsule containing the combination of carbonyl iron, vitamin C, and ginger.

11. The nutritional supplement of claim 1, wherein the iron supplement is one or more of a tablet, a capsule, a suspension, a gum, or an effervescent tablet.

12. The nutritional supplement of claim 1, wherein the ginger is a ginger extract.

13. The nutritional supplement of claim 12, wherein the ginger extract is combined with the carbonyl iron comprising of a ratio of 10:1.

14. The nutritional supplement of claim 13, wherein the ginger extract is combined with the carbonyl iron comprising of a ratio of 5:1.

15. The nutritional supplement of claim 13, wherein the ginger extract is configured as a ginger component within the iron supplement to produce additional gastric acid.

16. The nutritional supplement of claim 1, wherein the vitamin C is water-soluble and a reducing agent.

17. The nutritional supplement of claim 1, wherein the vitamin C is an amount between 30 milligrams (mg), and 70 mg.
